(19) United States
(12) Patent Application Publication
(10) Pub. No.: US 2020/0114068 A1
(43) Pub. Date: Apr. 16, 2020

(54) INJECTOR DEVICE

(71) Applicant: Novartis AG, Basel (CH)

(72) Inventors: Alain Schmidlin, Basel (CH); Mario Iobbi, Basel (CH); Erich Studer, Basel (CH); Andrew Bryant, Basel (CH); Chinmay Deodhar, Pune (IN); Rajan Patel, Menlo Park, CA (US)

(21) Appl. No.: 16/494,233

(22) PCT Filed: Feb. 1, 2018
(86) PCT No.: PCT/EP2018/052505

§ 371 Date: Sep. 13, 2019

Related U.S. Application Data
(60) Provisional application No. 62/471,987, filed on Mar. 16, 2017.

(30) Foreign Application Priority Data
Mar. 31, 2017 (EP) 17164073.3

(51) Int. Cl. A61M 5/145

(52) U.S. Cl. CPC A61M 5/1454; A61M 2005/1585

(57) ABSTRACT

A driving mechanism (100) for an injector device comprises a coil spring (10) having a proximal end (12) and a distal end (14), a spring guide (20) adapted to guide a movement of the coil spring (10), and an actuator (50, 52, 54, 56, 58, 60) adapted to cause a force on the proximal end (12) of the coil spring (10), wherein the spring guide (20) comprises a curved part (22) having a curved shape. An injector device comprises a driving mechanism (100) and a needle ejection mechanism comprising a hollow skin needle, wherein the driving mechanism (100) is adapted to cause a dispensing of a drug from a cartridge (70) through the hollow skin needle and an ejection of the hollow skin needle from the injector device.

The invention relates to an injector device, in particular, an injector device with a driving mechanism that is adapted to be installed in a hand-held injector device.

Hand-held injector devices require a compact size so that they may be held and operated by the hands of a user. On the other hand, there is a trend that more and more components and mechanisms are installed in hand-held injector devices. For example, hand-held injector devices may include a needle ejection and rejection mechanism, an automatic door opening and closing mechanism, a cartridge piercing mechanism, etc. However, problems may arise if all such components and mechanisms are to be installed in a single hand-held injector device. In particular, problems may arise if all such components and mechanisms are to be installed in an injector device that is designed to be placed on the skin of a human or animal body, e.g., an injector device having a relatively small skin-contact surface.

U.S. Pat. No. 7,749,194 B2 concerns a medicament delivery mechanism comprising a compressed gas container containing a compressed gas. Upon puncturing of a gas port, the compressed gas escapes from the container and applies gas pressure to a medicament actuator. The medicament actuator interfaces with a medicament storage assembly comprising a plurality of vials containing a liquid medicament and a plurality of plungers, each of which is capped with a piston which can move within a corresponding vial. Within the medicament delivery mechanism, the gas container is arranged in parallel to the medicament actuator and a gas channel connects the gas container and the medicament actuator.

U.S. Pat. No. 5,957,889 concerns displacement systems for controlled infusion of a liquid from a cartridge of the kind comprising a tubular vessel, which is at a rear end closed by a piston which is forced by a piston rod into the tube to press out the liquid through an outlet arranged at a front end of the tube. The piston rod may be a flexible helix with narrowly adjacent turns of windings. This piston rod is guided by a piston rod guide.

GB 2 145 795 A concerns a mechanical transmission which transfers power from a motor or to a piston in an aspirator, and includes an elongate transmission element formed as a single or double spiral of tightly wound wire.

WO 2005/072795 A2 concerns a medicine dispensing device comprising a housing, adhesive means for attaching the housing to the skin of a user of the device, a cylindrical medicine container in the housing and incorporating a piston and having an open distal end and a proximal end closed by a closure body made of a material that can be perforated by a needle, a piston rod with a longitudinally extending first screw thread on the outer surface thereof for displacing the piston towards the closed proximal end, a ratchet wheel with a central aperture for receiving the piston rod and provided with a second screw thread meshing with the first screw thread such that rotation of the ratchet wheel in a first rotational direction will displace the piston, a pivotable element pivotable around an axis adjacent the ratchet wheel and provided with a pawl for engaging the teeth of the ratchet wheel such that pivoting of the pivotable plate in a second rotational direction rotates the ratchet wheel in the first rotational direction, a rod spring adapted and arranged for applying a spring force to the plate at a first point spaced from the axis for pivoting the plate in the second rotational direction, and a battery powered actuator for rotating the pivotable plate in a third rotational direction opposite the second rotational direction.

The invention is directed at the object of providing an improved injector device having a compact size.

This object is addressed by an injector device as defined in claim 1.

The driving mechanism for an injector device comprises a coil spring having a proximal end and a distal end, a spring guide adapted to guide a movement of the coil spring, and an actuator adapted to cause a force on the proximal end of the coil spring, wherein the spring guide comprises a curved part having a curved shape.

The terms “proximal” and “distal” in the sense of the present disclosure relate to the movement direction of the actuator in order to cause a force on the proximal end of the coil spring. Accordingly, when the actuator causes a force on the proximal end of the coil spring, the proximal end of the coil spring is moved in a distal direction.

The coil spring, which may also be called a “slinky spring”, may be any kind of coil spring that is adapted to store and release mechanical energy. In case the coil spring is uncompressed or only partly compressed before the actuator causes the force on the proximal end of the coil spring, upon exertion of the force on the proximal end of the coil spring, the coil spring is compressed further or compressed to its maximum before it is moved along the spring guide.

Preferably, the coil spring is made of a steel having a high carbon content, for example, made of a music wire. Music wire provides the advantages of a high tensile strength and a high elastic limit. Moreover, music wire can withstand high stresses under repeated loadings. It also possible that the coil spring is replaced by more than one coil spring and the spring guide is adapted to guide more than one coil spring.

The spring guide may be any kind of structure that allows a movement of the coil spring along a predefined movement path. In one embodiment, the spring guide has a tubular shape with an inner diameter that fits the outer diameter of the coil spring. Preferably, the inner diameter of the spring guide and the outer diameter of the coil spring are selected such that only a minimal resistive force occurs when the coil spring slides in the spring guide, however, the spring guide ensures a safe guidance of the coil spring such that the coil spring does not twist and/or gets stuck in the spring guide. Alternatively, the spring guide may also have a cylindrical cross section with an outer diameter that fits the inner diameter of the coil spring so that the coil spring is provided around the spring guide.

The actuator may be any kind of force-generating element that causes a force on the proximal end of the coil spring, i.e., a force that is higher than the resistive force that is necessary for moving the coil spring through or along the spring guide. Preferably, the actuator is adapted to cause a constant force on the proximal end of the coil spring.

In one embodiment, the actuator comprises an electrical motor. Specifically, the electric motor may be a single direct current (DC) electric motor. Preferably, the motor is adapted to provide a relatively high torque.

The curved part of the spring guide may be substantially U-shaped so that a force that is applied to the proximal end of the coil spring in a first direction causes a force at the distal end of the coil spring in a second direction that is substantially opposite to the first direction. Moreover, the curved part may have a shape of an arc, in particular, a shape of a circular arc. Preferably, the circular arc may have a central angle in the range from 135° to 225°. Thus, it is possible to arrange the actuator and an element that receives the force from the actuator by the distal end of the coil spring at proximate locations, which makes it possible to install these elements in a hand-held injector device having a compact size, for example, a device have a shape like a computer mouse.

In one embodiment, the spring guide comprises a first straight part which extends along a first longitudinal axis and a second straight part which extends along a second longitudinal axis, wherein the curved part is provided between the first straight part and the second straight part. Alternatively, the spring guide may also comprise only one of the first straight part and the second straight part. Moreover, each of the first straight part and the second straight part may have a relatively small length along the first longitudinal axis and the second longitudinal axis as compared to the length of the curved part.

In a preferred embodiment, the first longitudinal axis and the second longitudinal axis are substantially parallel to each other. Herein, the term “substantially parallel” comprises an angle in the range from 0° to 20° between the point of intersection of the first longitudinal axis and the second longitudinal axis.

Further preferably, the first longitudinal axis and the second longitudinal axis lie in a same plane. Alternatively, it is also possible that the first longitudinal axis and the second longitudinal axis lie in two different planes which cross each other at an angle in the range from 0° to 20°.

The driving mechanism may further comprise a plunger adapted to receive a force by the distal end of the coil spring and cause a force on a piston of a cartridge. For example, the cartridge may be a drug storage cartridge comprising a dispensing port, a drug storage chamber containing a liquid drug, and the piston which is movable within the cartridge so that the drug can be forced out of the dispensing port when the piston is pushed towards the distal end of the cartridge. Accordingly, it is possible to locate the actuator in proximity to the cartridge so that the driving mechanism may be installed in a hand-held injector device having a compact size. For moving the piston within the cartridge, the actuator may be configured to cause a force on the proximal end of the coil spring that is larger than the force for compressing the coil spring, the resistive force for moving the coil spring in or along the spring guide, and the resistive force for moving the piston within the cartridge.

In a preferred embodiment, the actuator comprises a leadscrew having a male thread, a motor adapted to cause a rotation of the leadscrew, a nut comprising a female thread which is provided around the male thread, wherein the nut is adapted to cause the force on the proximal end of the coil spring, and a nut guide adapted to guide a movement of the nut when the motor causes the rotation of the leadscrew. Accordingly, rotation of the leadscrew by the motor in clockwise and counterclockwise directions cause movement of the nut in directions towards and away from the proximal end of the coil spring. Preferably, the leadscrew extends along a direction that is substantially parallel to at least one of the first and second longitudinal axes. Further preferably, a surface of the nut is adapted to push the proximal end of the coil spring.

In order to effectively release the force by the distal end of the coil spring, the coil spring may have a larger free length than the length of the spring guide. The larger free length of the coil spring than the length of the spring guide may ensure that the plunger can sufficiently push the cartridge's piston so that all or most of the liquid drug stored in the cartridge can be dispensed through the dispensing port of the cartridge.

Moreover, in order to enable the nut to cause further actions than the force on the proximal end of the coil spring, the coil spring may have a larger free length than the length of the leadscrew. For example, when the nut is moved towards the spring guide, before causing a force on the piston of the cartridge, the nut may trigger an ejection of a hollow skin needle by a needle ejection mechanism. The cartridge may be connected to the hollow skin needle such that the liquid drug stored in the cartridge may be dispensed through the hollow skin needle into a human or animal body. For this, the nut may comprise a protrusion that is adapted to trigger the ejection of the hollow skin needle.

In order to ensure a precise start of the driving mechanism, the driving mechanism may further comprise a switch, wherein the motor is adapted to cause a movement of the nut and a stopping of the movement of the nut when the nut activates the switch. In particular, the motor may be adapted to cause a movement of the nut in a direction opposite to the direction where the nut causes the force on the proximal end of the coil spring and cause a stopping of the movement of the nut when the nut activates the switch. For example, when an injector device comprising the driving mechanism is powered on, by means of the switch, the nut may be moved to and stopped at a home position where the driving operation of the driving mechanism starts. By means of this calibration, it can be ensured that the driving mechanism always starts at the same position.

In a further preferred embodiment, the motor is adapted to cause a movement of the nut in a direction opposite to the direction where the nut causes the force on the proximal end of the coil spring, thereby causing a release of a door lock. In this embodiment, the motor has a double function of providing a force on the coil spring and causing a release of a door lock. For example, the door lock may be a lock for a door which allows a cartridge to be loaded into the injector device. In this case, before the nut causes the force on the proximal end of the coil spring, the nut may be moved in the opposite direction so that the door lock may be released, for example, in order to exchange an expired cartridge. The door lock may additionally ensure that the door cannot be opened after the driving operation by the driving mechanism has started.

Further preferably, the actuator may be adapted to cause an ejection of a hollow skin needle and thereafter a dispensing of a drug from the cartridge trough the hollow skin needle. For example, when the nut is moved in the direction towards the proximal end of the coil spring, during the movement path, it may first trigger an ejection of the hollow skin needle, (for example, by releasing at least one spring that causes an ejection of the hollow skin needle), and thereafter, by causing a force on the proximal end of the coil spring, cause a force on a piston of a cartridge by the distal end of the coil spring so that a drug stored in the cartridge is forced out of a dispensing port of the cartridge and through the hollow skin needle. In this embodiment, the actuator provides a double function of ejecting the hollow skin needle and thereafter dispensing the drug from the cartridge. Preferably, this double function can be provided in case the coil spring has a larger free length than the length of the leadscrew. Moreover, since the dispensing of the drug from the cartridge may be caused after the ejection of the hollow skin needle, it can be prevented that the drug stored in the cartridge may get clogged in the hollow skin needle. Still further, a triple function may be provided by the nut in case all of the release of the door lock, the ejection of the hollow skin needle, and the dispensing of the drug from the cartridge are triggered by the movement of the nut.

The invention further concerns an injector device comprising a driving mechanism and a needle ejection mechanism comprising a hollow skin needle, wherein the driving mechanism is adapted to cause a dispensing of a drug from a cartridge through the hollow skin needle and an ejection of the hollow skin needle from the injector device. For example, after the actuator has caused the hollow skin needle to be ejected from the injector device, the actuator may cause a dispensing of a drug from the cartridge through the hollow skin needle.

Preferred embodiments of the invention will now be described in further detail with reference to the appended drawings, wherein:

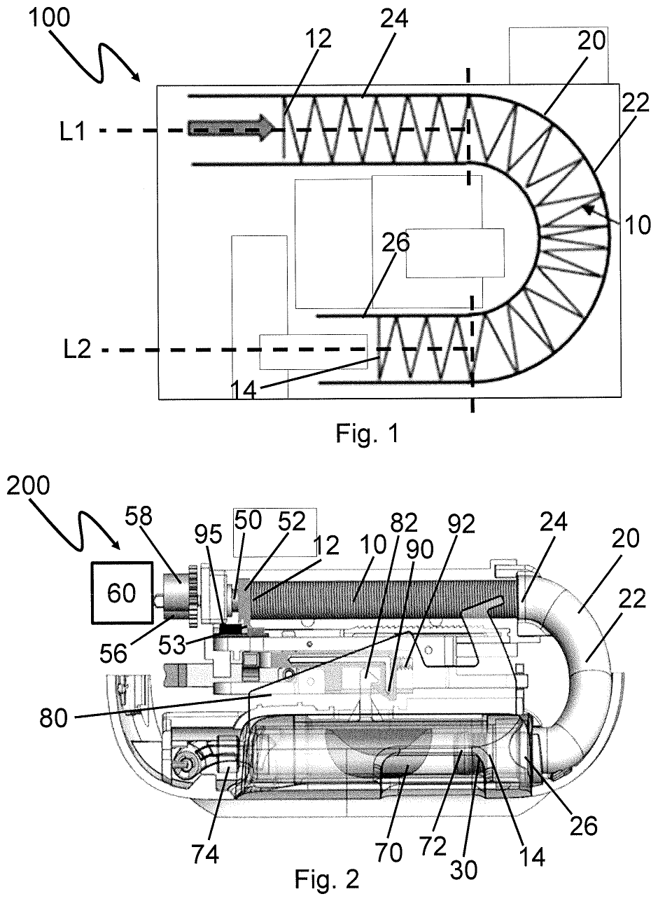

FIG. 1 schematically shows a driving mechanism according to a first embodiment;

FIGS. 2 to 6 schematically show an injector device comprising a driving mechanism according to a second embodiment;

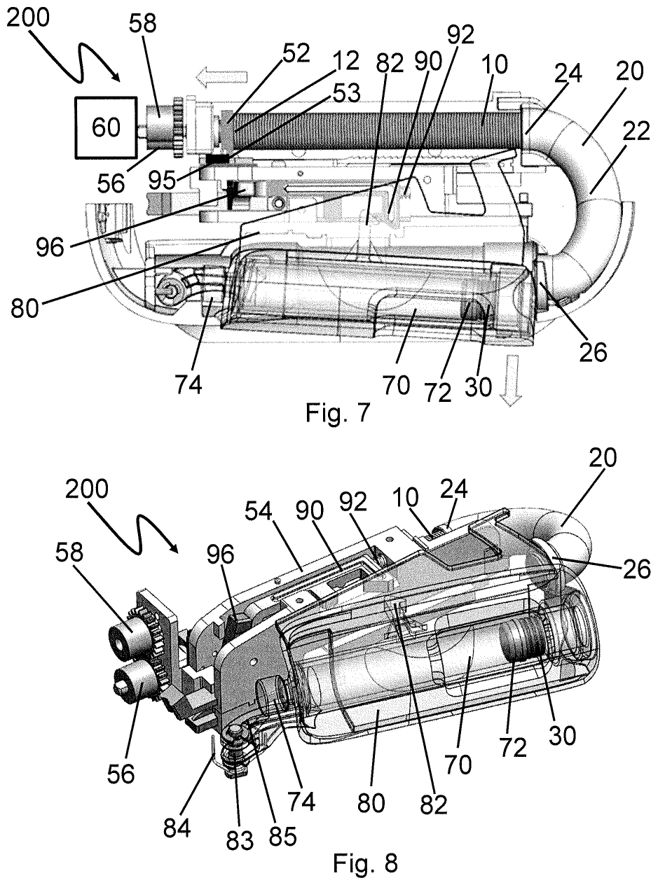

FIG. 7 schematically shows the injector device according to second embodiment with an opened cartridge container;

FIG. 8 shows a perspective view of an injector device with a driving mechanism and a cartridge container; and

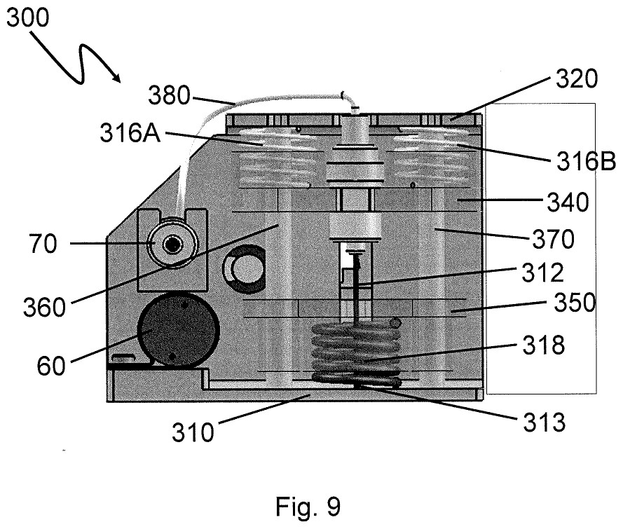

FIG. 9 schematically shows an injector device with a needle ejection and retraction mechanism according to a third embodiment.

FIG. 1 schematically shows a driving mechanism 100 according to a first embodiment. The driving mechanism 100 comprises a spring guide 20 and a coil spring 10. The spring guide 20 consists of a curved part 22, a first straight part 24 and a second straight part 26. The curved part 22 is provided between the first straight part 24 and the second straight part 26. The first straight part 24 extends along a first longitudinal axis L1, and the second straight part extends along a second longitudinal axis L2. The first longitudinal axis L1 and the second longitudinal axis L2 are substantially parallel to each other. The coil spring 10 comprises a proximal end 12 and a distal end 14, and is adapted to be moved and compressed within the spring guide 20. For this, the coil spring 10 has an outer diameter that matches the inner diameter of the spring guide 20 so that the coil spring 10 can slide through the spring guide 20. The coil spring 10 is made of a steel having a high carbon content, e.g., made of a music wire. The spring guide 20 is made of a rigid plastic material.

When a force is exerted on the proximal end 12 of the coil spring 10 (as indicated by the arrow), the coil spring 10 is forced through the spring guide 20 so that the force that is caused on the proximal end 12 of the coil spring 10 is exerted at the distal end 14 of the coil spring 10, i.e., the direction of the force (as indicated by the arrow) can be reversed. By changing the curvature of the spring guide 20, i.e., installing a differently shaped curved part 22, it is possible to adjust the angle at which the force that is caused at the proximal end 12 of the coil spring 10 is diverted at the distal end 14 of the coil spring 10.

FIGS. 2 to 6 schematically show an injector device 200 comprising a driving mechanism according to a second embodiment. The driving mechanism 100 schematically shown in FIG. 1 may be installed in the second embodiment according to FIGS. 2 to 6. Accordingly, the same reference numbers concern the same elements and any repeated explanation thereof is omitted. Moreover, for the sake of clarity, elements which are shown in FIGS. 2 to 6, however, which are not directly relevant for the operation of the driving mechanism are not explained in detail.

The driving mechanism according to the second embodiment differs from the driving mechanism according to the first embodiment in that the first straight part 24 and the second straight part 26 of the spring guide 20 have a relatively small length compared to the length of the curved part 22. Moreover, it is also possible to omit the first straight part 24 and/or the second straight part 26.

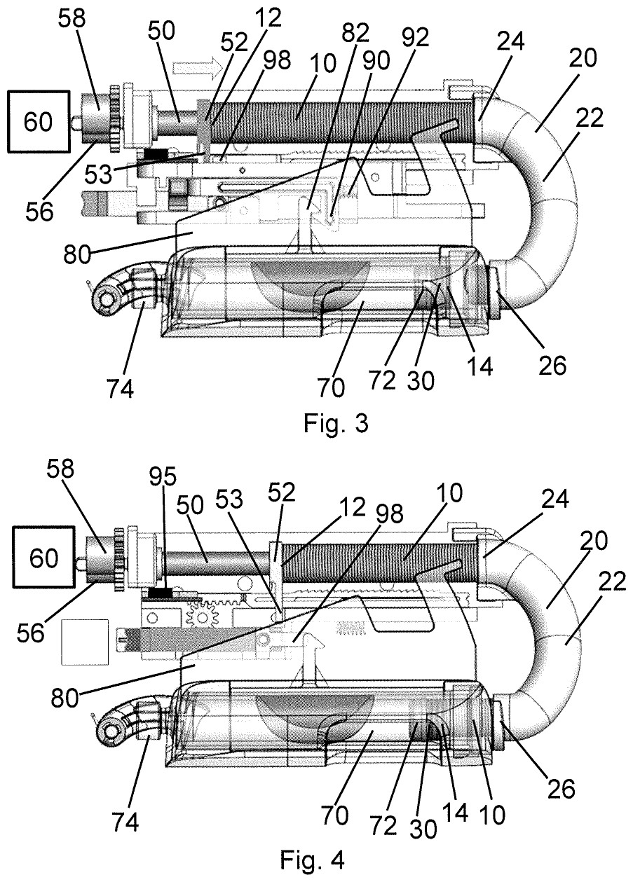

FIG. 2 shows the injector device 200 at a start state of the driving mechanism before the driving mechanism starts its operation for driving the coil spring 10. FIGS. 3 to 6 illustrate how the driving mechanism operates after the start state.

The injector device 200 shown in FIGS. 2 to 6 comprises a leadscrew 50 having a male thread (not shown in FIGS. 2 to 6). On the leadscrew 50, a nut 52 is provided. The nut 52 comprises a female thread that fits the male thread of the leadscrew 50 and a protrusion 53. When the leadscrew 50 is rotated, the nut 52 is moved along the leadscrew 50. For this, a nut guide (for example, a wall element that extends parallel to the leadscrew 50, not shown in FIGS. 2 to 6) guides the nut 52. When the nut 52 is moved along the leadscrew 50 towards the spring guide 20, as indicated by the arrow in FIG. 3, a surface of the nut 52 abuts the proximal end 12 of the coil spring 10 and pushes the coil spring 10 through the spring guide 20.

At the proximal end of the leadscrew 50, a first gear 56 is provided. The first gear 56 is engaged with a second gear 58. A motor 60 rotates the second gear 58, which causes a rotation of first gear together with the leadscrew 50 and the movement of the nut 52 along the leadscrew 50. The motor 60 is a direct current electric motor that is controlled by a motor control unit (not shown in FIGS. 2 to 6). The motor 60 is adapted to provide a relatively high torque. The speed of the motor 60 is controlled by the motor control unit via a pulse-width modulation (PWM) control scheme. Thus, a constant movement of the nut 52 along the leadscrew 50 can be provided.

At the distal end 14 of the coil spring 10, a plunger 30 is provided. The plunger 30 may be connected to the distal end 14 of the coil spring 10 and moved by the movement of the coil spring 10. The injector device 200 further comprises a cartridge container 80, which is adapted to receive a drug storage cartridge 70. The cartridge container 80 comprises a door lock hook 82 and has a door function which allows an opening and a closing of the cartridge container 80 so that the cartridge 70 can be inserted into the injector device 200. The cartridge container 80 further comprises a window through which the cartridge 70 can be seen from the outside. The door lock hook 82 is adapted to be engaged with a second hook 90 so that the cartridge container 80 can be locked in the closed state shown in FIGS. 2 to 6. Furthermore, a spring 92 is provided which pushes the second hook 90 towards the door lock hook 82. Thus, the second hook 90 is installed in the injector device 200 and is adapted to be movable in two directions.

The drug storage cartridge 70 stores a dispensable drug and comprises a piston 72 and a dispensing port 74. The piston 72 is movable within the cartridge so that when the plunger 30 pushes the piston 72 from an initial proximal position in the distal direction, the drug stored in the cartridge 70 is forced through the dispensing port 74 out of the cartridge 70. Furthermore, the dispensing port 74 may be sealed by a septum (not shown in FIGS. 2 to 6), which is pierced before the drug can be forced out of the cartridge 70.

To explain the operation of the injector device 200 in more detail, when the nut 52 is moved from the home position shown in FIG. 2 along the leadscrew 50 towards the spring guide 20 (as shown in FIG. 3), it pushes the proximal end 12 of the coil spring 10 (together with the coil spring 10) towards the spring guide 20. In the state shown in FIG. 3, the distal end 14 of the coil spring 10 has not yet caused the plunger 30 to push the piston 72. When the nut 52 shown in FIG. 3 is moved to the state shown in FIG. 4, the protrusion 53 of the nut 52 pushes a lever 98 of a needle ejection mechanism, which causes a release of at least one ejection spring that causes an ejection of a hollow skin needle (not shown in FIGS. 2 to 6). After the pushing of the lever 98 by the protrusion 53 shown in FIG. 3, as can be seen from FIG. 4, the lever 98 is rotated by a 90° in a clockwise direction in order to allow the nut 52 with the protrusion 53 to be moved back to the home position shown in FIG. 2.

For detecting the home position of the nut 52 shown in FIG. 2, the injector device 200 comprises a switch 95 that can be activated by the nut 52. In particular, when the nut 52 is moved along the leadscrew 50 and activates the switch 52, the movement of the nut 52 is stopped since the nut 52 is located at its home position, as shown in FIG. 2. In particular, upon an initial power-on of the injector device 200, by means of the switch 52, the nut 52 can be moved to its home position. Thus, it can be ensured that the driving mechanism always starts with the nut 52 at the same location.

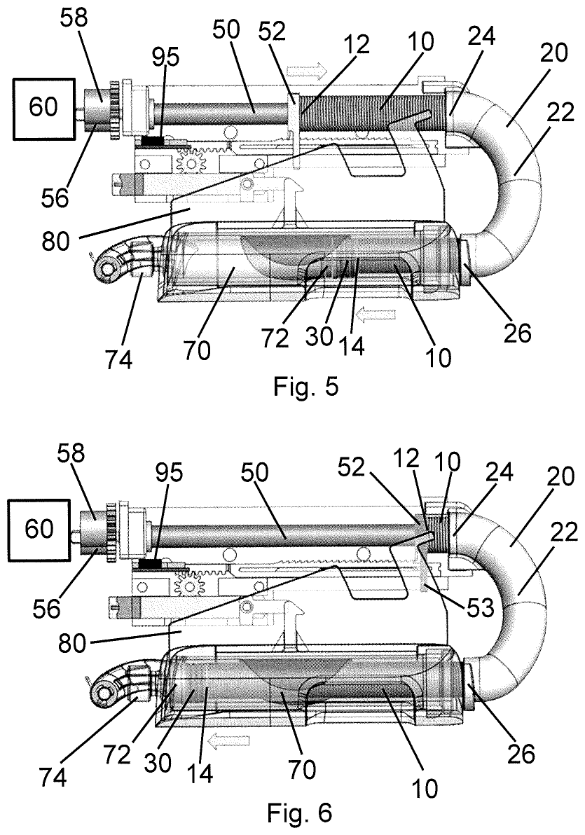

After the triggering of the hollow skin needle by the movement of the nut 52 from the state shown in FIG. 3 to the state shown in FIG. 4, the distal end 14 of the coil spring 10 together with the plunger 30 pushes the piston 72 in the distal direction of the cartridge 70 so that the drug is forced out of the cartridge 70. The further movement of the nut 52 towards the spring guide 20 and the further dispensing of the drug out of the cartridge 70 are illustrated in FIG. 5.

FIG. 6 shows the end of the drug dispending process, at which the plunger 30 has pushed the piston 72 to the distal end of the cartridge 70 so that all or most of the drug is dispensed through the dispensing port 74. Specifically, from the dispensing port 74, the drug is dispensed through the hollow skin needle into a human or animal body (not shown in FIGS. 2 to 6).

The injector device 200 according to the second embodiment provides a double function in that an ejection of a hollow skin needle and a dispensing of a drug from the cartridge 70 are caused by the movement of the nut 52. Specifically, since the hollow skin needle is first ejected and thereafter the drug is dispensed from the cartridge 70 via a conduit through the hollow skin needle, it can be prevented that the drug gets clogged in the conduit and the hollow skin needle.

FIG. 7 schematically shows the injector device 200 according to second embodiment with the cartridge container 80 being opened. When the cartridge container 80 is completely opened, the cartridge 70 can be loaded into the cartridge container 80.

When the nut 52 is moved from the home position shown in FIG. 3 (at which the cartridge container 80 is closed) towards the first gear 56 and the second 58, as indicated by the upper arrow in FIG. 7, the protrusion 53 of the nut 52 triggers a pivoted lock wheel 96. The lock wheel 96 pushes the second hook 90 towards the spring 92, which results in disengagement of the second hook 90 and the door lock hook 82. Thus, the cartridge container 80 can be opened, as indicated by the lower arrow shown in FIG. 7. Thereafter, when the cartridge container 80 is closed, the second hook 90 snaps and engages with the door lock hook 82 so that the cartridge container 80 cannot be opened anymore, as shown in FIG. 2

Thus, in addition to the two functions of the nut 52 described above, the nut 52 can cause a third function of releasing a door lock, i.e., allow the cartridge container 80 to be opened.

FIG. 8 shows a perspective view of the injector device 200 according to FIGS. 2 to 7. In FIG. 8, certain elements shown in FIGS. 2 to 7 have been omitted, like the leadscrew 50 and the motor 60. From FIG. 8, it can be seen in more detail where the first gear 56 and the second gear 58 are located and how they interact with each other. Moreover, FIG. 8 shows a nut guide 54 which is realized as a wall along which the nut 52 is moved and which guides the nut 52 when the leadscrew 50 is rotated (not shown in FIG. 8).

FIG. 8 further shows the lock wheel 96. Upon initiation by the nut 52 (not shown in FIG. 8), the lock wheel 96 pushes the second hook 90 towards the spring 92, which results in disengagement of the second hook 90 and the door lock hook 82 so that the cartridge container 80 can be opened.

FIG. 8 further shows how the cartridge container 80 automatically opens when the second hook 90 and the door lock hook 82 disengaged. For this, a pin 83 is provided at a hinge 85 of the cartridge container 80. A torque exerted by a torsion spring 84 forces the cartridge container 80 to be rotated around the pin 83 to the opened position shown in FIG. 8.

FIG. 9 schematically shows an injector device 300 with a needle ejection and retraction mechanism. The injector device 300 may comprise the elements of the first and second embodiments according to FIGS. 1 to 8. Thus, the same reference numbers relate to the same elements and any repeated explanation thereof is omitted.

Specifically, FIG. 9 shows how a needle ejection and retraction mechanism may be s installed in the injector device 300 and how a hollow skin needle 312 may be ejected into the skin of a human or animal body before the drug is dispensed from the cartridge 70 via a conduit 380 through the hollow skin needle 312.

The needle ejection and retraction mechanism is provided in the housing of the injector device 300, which comprises a base plate 310 and a top plate 320. Between the base plate 310 and the top plate 320, two vertical rods 360 and 370 are foreseen. The outer surface of the base plate 310 is adapted to be placed on the skin of a human or animal body.

The injector device 300 comprises the hollow skin needle 312 having a needle tip 313 which is adapted to pierce the skin of a human or animal body, two compressed first springs 316A and 316B for ejecting the skin needle 312 out of the injector device 300, and a second compressed spring 318 for retracting the skin needle 312 back into the injector device 300. The two first springs 316A and 316B extend in parallel to an axis along which the skin needle 313 is ejected, and are provided between the top plate 320 and a first support element 340 which is connected to a proximal end of the skin needle 312. Specifically, movement of the nut 52 (not shown in FIG. 9) releases the spring forces of the two compressed first springs 316A and 316B so that the skin needle 312 is caused to be pushed by the two first springs 316A and 316B out of the injector device 300. For example, the movement of the nut 52 may cause a lever (not shown in FIG. 9) holding the first springs 316A and 316B to be removed so that the two first springs 316A and 316B cause an ejection of the first support element 340 together with the skin needle 312.

Upon ejection of the skin needle 312, the needle tip 313 protrudes from the outer surface of the base plate 310 (not shown in FIG. 9). The second spring 318 is provided between the base plate 310 and a second support element 350, and is adapted to retract the skin needle 312 back into the injector device 300. FIG. 9 further shows the drug storage cartridge 70 which is connected via the conduit 80 to the proximal end of the hollow skin needle 312. Thus, the conduit 80 is adapted to establish a fluid path between the proximal end of the skin needle 312 and the cartridge 70.

Since the driving mechanism comprises the coil spring 10 and the curved spring guide 20 (not shown in FIG. 9), the cartridge 70 can be located close to the motor 60 so that the size of the injector device 300 can be reduced.

Although FIG. 9 illustrates the injector device 300 comprising a needle ejection and retraction mechanism, the driving mechanism may also be installed in an injector device only comprising a needle ejection mechanism. For this, the second spring 318 may be removed.

1. An injector device comprising a driving mechanism, the driving mechanism comprising
a coil spring having a proximal end and a distal end;
a spring guide adapted to guide a movement of the coil spring; and
an actuator adapted to cause a force on the proximal end of the coil spring, wherein
the spring guide comprises a curved part having a curved shape, and a needle ejection mechanism comprising a hollow skin needle, wherein
the actuator is adapted to cause an ejection of the hollow skin needle and thereafter a dispensing of a drug from a cartridge inserted in the injector device through the hollow skin needle.

2. The injector device according to claim 1, wherein
the spring guide comprises a first straight part which extends along a first longitudinal axis and a second straight part which extends along a second longitudinal axis, wherein the curved part is provided between the first straight part and the second straight part.

3. The injector device according to claim 2, wherein
the first longitudinal axis and the second longitudinal axis are substantially parallel to each other.

4. The injector device according to claim 2, wherein the first longitudinal axis and the second longitudinal axis lie in a same plane.

5. The injector device according to claim 1, wherein the spring guide has a tubular shape with an inner diameter that fits the outer diameter of the coil spring.

6. The injector device according to claim 1, wherein the coil spring has a larger free length than the length of the spring guide.

7. The injector device according to claim 1, further comprising
a plunger adapted to receive a force by the distal end of the coil spring and cause a force on a piston of a cartridge.

8. The injector device according to claim 1, wherein the coil spring is made of steel having a high carbon content.

9. The injector device according to claim 1, wherein the actuator comprises
a leadscrew having a male thread;
a motor adapted to cause a rotation of the leadscrew;
a nut comprising a female thread which is provided around the male thread, wherein the nut is adapted to cause the force on the proximal end of the coil spring; and
a nut guide adapted to guide a movement of the nut when the motor causes the rotation of the leadscrew.

10. The injector device according to claim 9, wherein the coil spring has a larger free length than the length of the leadscrew.

11. The injector device according to claim 9, further comprising
a switch, wherein
the motor is adapted to cause a movement of the nut and a stopping of the movement of the nut when the nut activates the switch.

12. The injector device according to claim 9, wherein the motor is adapted to cause a movement of the nut in a direction opposite to the direction where the nut causes the force on the proximal end of the coil spring, thereby causing a release of a door lock.

13. The injector device according to claim 1, wherein the actuator comprises an electric motor.
